(19) United States
(12) Patent Application Publication
(10) Pub. No.: US 2010/0092519 A1
(43) Pub. Date: Apr. 15, 2010

(54) COMPOSITIONS COMPRISING POLYSACCHARIDE CONJUGATES AND THEIR USE AS VACCINES

(75) Inventors: Andrew Richard Gorringe, Wiltshire (GB); Michelle Finney, Wiltshire (GB); Subhash V. Kapre, Pune (IN); Akshay Goel, Pune (IN)

Correspondence Address: ROYLANCE, ABRAMS, BERDO & GOODMAN, L.L.P., 1300 19TH STREET, N.W., SUITE 600, WASHINGTON,, DC, 20036, US

(73) Assignees: HEALTH PROTECTION AGENCY, Salisbury, Wiltshire (GB); SERUM INSTITUTE OF INDIA LIMITED, Pune (IN)

(21) Appl. No.: 12/527,725

(22) PCT Filed: Feb. 20, 2008
(86) PCT No.: PCT/GB08/50110

§ 371 Date: Oct. 6, 2009

(30) Foreign Application Priority Data
Feb. 21, 2007 (GB) 0703369.9

(51) Int. Cl. A61K 39/095; A61K 35/74; A61P 35/04

(52) U.S. Cl. 424/250.1; 424/780

(57) ABSTRACT

The present invention is in the field of combination therapies, including vaccine compositions, which comprise polysaccharide-protein conjugates and outer membrane vesicles (OMVs) from commensal bacteria, particularly commensal Neisseria.

The present invention is in the field of combination therapies which comprise polysaccharide-protein conjugates and outer membrane vesicles (OMVs) from commensal bacteria.

Vaccines that are based on a conjugate of a polysaccharide (such as, by way of example, capsular polysaccharides or lipopolysaccharides from bacteria) conjugated to a protein carrier are well known in the art (e.g., Jones, 2005).

The use of outer membrane vesicles of Neisseria lactamica (a commensal Neisseria) as a vaccine against meningococcal disease caused by N. meningitidis (a pathogenic Neisseria) has been discussed in the art; N. lactamica OMVs have been demonstrated to protect against lethal challenge in a mouse model of meningococcal disease (Gorringe, 2005; Oliver 2002; WO00/50074). In addition, outer membrane vesicles from N. meningitidis have been used in vaccines against meningococcal disease, as discussed in more detail below. Outer membrane vesicles from N. meningitidis and N. lactamica have also been used in a vaccine blend (WO 03/051379).

N. meningitidis is the agent that causes meningococcal meningitis and is of particular importance as a worldwide health problem. It is also responsible for meningococcal septicaemia. N. meningitidis is classified on the basis of the capsular polysaccharide, giving several serogroups, including A, B, C, Y and W135 (Harrison, 2006).

It is known to provide vaccines based on the capsular polysaccharide of N. meningitidis, some of which use a conjugate in which the capsular polysaccharide is conjugated to a protein moiety, such as tetanus toxin, which increases the immunogenicity of the polysaccharide (Jones, 2005). Aaberge et al (2005) proposed a bivalent vaccine for parenteral administration in which a conjugate of meningococcal serogroup C capsular polysaccharide is mixed with OMVs from N. meningitidis serogroup B. The immune response generated by this vaccine was reported to be similar to the immune response of each of its constituent components when administered individually, thus there was no enhancement of immune response to the capsular polysaccharide when mixed with N. meningitidis OMVs. Sardinas et al (2006) reported that meningococcal and N. lactamica OMVs were equally effective as mucosal adjuvants for Hepatitis B surface antigen protein (HBsAg).

Sierra et al (1991) proposed a vaccine against serogroup B meningococcal disease that is based on OMVs. In this vaccine, equal amounts of OMVs from serogroup B N. meningitidis are mixed with purified unconjugated capsular polysaccharide from serogroup C N. meningitidis and an immunological response to both serogroup B and serogroup C bacteria was produced.

Fukasawa et al (1999) proposed a bivalent vaccine in which meningococcal serogroup C capsular polysaccharide is chemically conjugated to outer membrane vesicles from N. meningitidis serogroup B.

Problems with combination vaccines based on conjugates have also been reported. For example, Finn and Heath (2005) review the problem of negative interactions caused between acellular pertussis vaccine and Haemophilus influenzae type B (Hib) conjugate vaccine.

There is a need for improved polysaccharide: protein conjugate-based therapies. Furthermore, there is a need for improved vaccines against meningococcal disease.

The present invention is based on the surprising finding that OMVs isolated from commensal Neisseria enhanced the immumological response to a polysaccharide:protein conjugate vaccine.

In a first aspect, the present invention provides a combination therapy comprising: (i) a conjugate of a polysaccharide and a carrier protein (polysaccharide: protein conjugate); and (ii) commensal Neisseria outer membrane vesicles (OMVs) for use as a medicament.

In another aspect, the invention provides a vaccine composition comprising (i) a conjugate of a polysaccharide and a carrier protein (polysaccharide:protein conjugate); and (ii) outer membrane vesicles (OMVs) from commensal Neisseria.

Combination therapy as used herein is intended to refer to (i) a conjugate of a polysaccharide and a carrier protein (polysaccharide:protein conjugate); and (ii) outer membrane vesicles (OMVs) from commensal Neisseria, which are for administration as individual components, or which are combined into a composition containing both (i) and (ii) prior to administration. Thus a combination therapy of the invention includes compositions containing both components and e.g. kits containing each of the components for separate, simultaneous or sequential administration. Such kits may include instructions for such administration.

Thus, the combination therapy of the invention may, in preferred embodiments, be administered in various ways. In one embodiment, the polysaccharide:protein conjugate and the outer membrane vesicles are administered simultaneously. In another embodiment, the conjugate and the outer membrane vesicles are administered separately. In another embodiment the conjugate and said outer membrane vesicles are administered in combination. In another embodiment the conjugate and the outer membrane vesicles are administered sequentially.

Any polysaccharide: protein conjugate may be used. Known polysaccharide:protein conjugate vaccines are described in Jones (2005). The polysaccharide may be from Gram negative bacteria, or from Gram positive bacteria. For example, the polysaccharide may be a capsular polysaccharide. The polysaccharide may be a lipopolysaccharide.

By way of example, the polysaccharide may be from Gram negative bacteria selected from the group consisting of: Escherichia coli, Francisella tularensis, Haemophilus influenzae, Klebsiella, Moraxella catarrhalis, Neisseria meningitidis, Porphyromonas gingivalis, Pseudomonas aeruginosa, Burkholderia cepacia, Salmonella typhi, Salmonella typhimurium, Salmonella paratyphi, Shigella dysenteriae, Shigella flexneri, Shegella sonnei and Vibrio cholera. The polysaccharide may be from Gram positive bacteria selected from the group consisting of: Enterococcus faecalis, Enterococcus faecium, Group A Streptococcus, Group B Streptococcus, Mycobacterium tuberculosis, Staphylococcus aureus, Staphylococcus epidermidis and Streptococcus pneumoniae.

It is preferred that the polysaccharide is from pathogenic Neisseria, preferably N. meningitidis. For example, the polysaccharide may be a Neisseria meningitidis capsular polysaccharide. The N. meningitidis may be selected from N. meningitidis serogroups A, C, W135, B and Y. Preferably, the N. meningitidis is selected from N. meningitidis serogroup A, C, W135 and Y (e.g., A and C; or A and W135; or A and Y; or C and W135; or C and Y; or W135 and Y; or A, C and W135; or A, C and Y; or C, W135 and Y; or A, Y and W135; or A, C, W135 and Y), more preferably the N. meningitidis is selected from N. meningitidis serogroup C and Y, most preferably the N. meningitidis is N. meningitidis serogroup C. Serogroup W135 is also known as serogroup W.

Multiple polysaccharides may also be used in this invention. In an embodiment of the invention, the combination therapy or vaccine may contain polysaccharides from N. meningitidis serogroups A, C, W135 and Y. Most preferred is a combination therapy or vaccine that contains polysaccharide conjugates from four serogroups, A, C, W135 and Y. Thus a pentavalent combination therapy or vaccine is provided comprising polysaccharide conjugates from serogroups A, C, W135 and Y together with the OMVs.

Other combinations are also provided in this invention and thus, in further embodiments, the invention provides combination therapies and vaccines which comprise combinations of polysaccharide conjugates from any combination of

N. meningitidis serogroups A, C, W135, B and Y. By way of example, the combination therapies or vaccines of the invention may comprise conjugates from: N. meningitidis serogroups A and C; N. meningitidis serogroups A and W135; N. meningitidis serogroups A and B; N. meningitidis serogroups A and Y; N. meningitidis serogroups C and W135; N. meningitidis serogroups C and B; N. meningitidis serogroups C and Y; N. meningitidis serogroups W135 and B; N. meningitidis serogroups W135 and Y; N. meningitidis B and Y; N. meningitidis serogroups A, C and W135; N. meningitidis serogroups A, C and B; N. meningitidis serogroups A, C and Y; N. meningitidis serogroups A, W135 and B; N. meningitidis serogroups A, W135 and Y; N. meningitidis serogroups A, B and Y; N. meningitidis serogroups C, W135 and B; N. meningitidis serogroups C, W135 and Y; N. meningitidis serogroups W135, B and Y; N. meningitidis serogroups A, C, W135 and B; N. meningitidis serogroups A, C, W135 and Y; N. meningitidis serogroups A, W135, B and Y; N. meningitidis serogroups A, C, B and Y; N. meningitidis serogroups C, W135, B and Y; or N. meningitidis serogroups A, C, W135, B and Y. Most preferred is A, C, W135 and Y.

There is also a further rare serogroup of N. meningitidis, serogroup X. Thus, in further embodiments of the combination therapy or vaccine of the invention, the polysaccharide conjugate may be from N. meningitidis, serogroup X. Polysaccharide from serogroup X may be combined in any of the other polysaccharide conjugates mentioned above. Thus, the invention further provides combination therapies and vaccines which comprise combinations of polysaccharide conjugates from any combination of N. meningitidis serogroups A, C, W135, B, Y and X, such as: A and X; C and X; W135 and X; B and X; or Y and X. By way of further example, the combination therapies or vaccines of the invention may comprise polysaccharide conjugates from: N. meningitidis serogroups X, A and C; N. meningitidis serogroups X, A and W135; N. meningitidis serogroups X, A and B; N. meningitidis serogroups X, A and Y; N. meningitidis serogroups X, C and W135; N. meningitidis serogroups X, C and B; N. meningitidis serogroups X, C and Y; N. meningitidis serogroups X, W135 and B; N. meningitidis serogroups X, W135 and Y; N. meningitidis X, B and Y; N. meningitidis serogroups X, A, C and W135; N. meningitidis serogroups X, A, C and B; N. meningitidis serogroups X, A, C and Y; N. meningitidis serogroups X, A, W135 and B; N. meningitidis serogroups X, A, W135 and Y; N. meningitidis serogroups X, A, B and Y; N. meningitidis serogroups X, C, W135 and B; N. meningitidis serogroups X, C, W135 and Y; N. meningitidis serogroups X, W135, B and Y; N. meningitidis serogroups X, A, C, W135 and B; N. meningitidis serogroups X, A, C, W135 and Y; N. meningitidis serogroups X, A, W135, B and Y; N. meningitidis serogroups X, A, C, B and Y; N. meningitidis serogroups X, C, W135, B and Y; or N. meningitidis serogroups X, A, C, W135, B and Y.

Using such a multivalent approach, it is possible to select appropriate polysaccharide conjugates according to the prevalent serogroups of N. meningitidis in a particular target population. For example, Stephens (2007) describes the prevalence of particular serogroups of N. meningitidis, with geographical locations. According to this analysis a preferred vaccine or combination therapy of the invention for Western Europe would comprise polysaccharide conjugates from serogroups B or C, or a combination of B and C; for Russia it would comprise polysaccharide conjugates from serogroups A, B or C, or combinations thereof, preferably A, B and C; for Asia it would comprise polysaccharide conjugates from serogroups A, B or C, or combinations thereof, preferably A, B and C; for Australia it would comprise polysaccharide conjugates from serogroups B or C, preferably B and C; for New Zealand it would comprise polysaccharide conjugates from serogroup B; for Africa it would comprise polysaccharide conjugates from serogroups A, W135, C or X, or combinations thereof, preferably A, W135, C and X; for South America it would comprise polysaccharide conjugates from serogroups B or C, preferably B and C; and for North America it would comprise polysaccharide conjugates from serogroups B, C or Y, or combinations thereof, preferably B, C and Y.

In addition to the possibility of a combination of polysaccharide:protein conjugates associated with the same disease (such as multiple polysaccharide:protein conjugates from N. meningitidis) together with the OMVs, the vaccine or combination therapy of the invention may comprise polysaccharide:protein conjugates associated with different diseases. Thus, for example, the combination therapy or vaccine of the invention may comprise a combination of polysaccharide:protein conjugates wherein the polysaccharides are from any combination of the Gram positive or Gram negative bacteria listed above. Preferred is a combination therapy or vaccine comprising OMVs, together with a polysaccharide: protein conjugate (wherein the polysaccharide is from Neisseria e.g., N. meningitidis) and at least one polysaccharide: protein conjugate from another Gram positive or Gram negative bacterium. Suitable polysaccharides and polysaccharide conjugates are described in Jones (2005). For example, preferred vaccines or combination therapies of the invention contain: a combination of an N. meningitidis polysaccharide conjugate plus a Hib polysaccharide conjugate (from Haemophilus influenzae), particularly Hib polysaccharide from type b Haemophilus influenzae; or a combination of an N. meningitidis polysaccharide and polysaccharide conjuagate from Streptococcus pneumoniae. Further details of suitable Haemophilus influenzae and Streptococcus pneumoniae polysaccharides and conjugates can also be found in Jones (2005).

Most preferred is the combination of commensal OMVs (especially N. lactamica OMVs) with a polysaccharide: protein conjugate from N. meningitidis serogroup C and a Hib polysaccharide:protein conjugate. Also preferred is the combination of commensal OMVs (especially N. lactamica OMVs) with a polysaccharide:protein conjugate from N. meningitidis serogroup C and a Streptococcus pneumoniae polysaccharide: protein conjugate

The combination therapy or vaccine of the invention may be for use in the treatment or prevention of infectious disease. In some embodiments the disease is meningococcal disease, such as meningococcal meningitis or meningococcal septicaemia. Preferably, said disease is meningococcal meningitis.

The combination therapy or vaccine of the invention may be for use in the treatment or prevention of cancer.

In further embodiments of the invention, the polysaccharide may be from a eukaryotic cell, e.g., may be a tumour-associated antigen such as by way of examples: B cell lymphoma (e.g., GM2, GD2); Breast tumour (e.g., GM2, globo H, TF(c), Le); Colon tumour (e,g. GM2, TF(c), STn(c), Ley, Tn, sialyl Le^{a}); Lung tumour (e.g, GM2, globo H, Le^{y}); Melanoma (e.g, GM2, GD2, GD3L, GD3); Neuroblastoma (e.g.,GM2, GD2, GD3L, polysialic acid); Ovary tumour (e.g., GM2, globo H, Tf(c), STn(c), Le); Prostate tumour (e.g, GM2, globo H, TF(c), Tn(c), STn(c), Le); Sarcoma (e.g, GM2, GD2, GD3L, GD3); Small cell lung cancer (e.g., GM2, FucGM1, globo H, polysialic acid, sialyl Le^{a}); Stomach (e.g., GM2, Ley, Le^{a}, sialyl Le^{a}). Further discussion can be found in Slovin et al 2005.

The OMVs may be from any commensal Neisseria, for example, the OMV may be from a commensal Neisseria selected from the group consisting of Neisseria lactamica, Neisseria sicca, Neisseria cinerea, Neisseria subflava, Neisseria elongata, Neisseria flavescens, and Neisseria polysaccharea. Preferably, the commensal Neisseria is Neisseria lactamica.

The OMVs may be isolated according to any method known in the art, for example as described in Frasch et al (2001). OMVs are discrete vesicles having a mean diameter of around 120 nm (WO06/00850) and typically within the range of 80-200nm. Preferred vesicle diameters are 90-175nm, 100-150nm or 110-130nm. OMVs are broken down by detergents, such as SDS.

The OMVs used in the present invention may be modified, e.g., to express one or more heterologous proteins, as described in Poolman and Berthet (2001) and O'Dwyer et al (2004).

Carrier proteins for use in conjugate vaccines are well known in the art. Examples are discussed in Jones et al (2005). Any suitable carrier protein may be used, for example the carrier protein may be selected from the group consisting of a toxoid, keyhole limpet haemocyanin, fimbrae, albumin, CRM197 and Pseudomonas aeruginosa exotoxin A. Preferably, the carrier protein is a toxoid, e.g., tetanus toxoid or diphtheria toxoid.

The combination therapy and vaccine of the invention may be administered parenterally, or orally or intranasally. Parenteral, such as subcutaneous, intramuscular or intradermal administration is preferred.

The combination therapy or vaccine of the invention may further comprise an adjuvant, or may be free, or substantially free, of adjuvant. Suitable adjuvants include the following: mineral salts e.g, aluminium hydroxide, aluminium phoshpate or calcium phosphate; oil emulsions and surfactants e.g., MF59 (microfluidised detergent stabilised oil in water emulsion), QS21 (purified saponin), Montanides (stabilised water in oil emulsion); particulates e.g., virosomes (unilamellar liposomal vehicles with influenza antigens), ISCOMS (structured complex of saponins and lipids), PLG (poly-lactic-co-glycolic acid), Chitosan; microbial (natural and synthetic) derivatives e.g., CpG oligodeoxynucleotides (ODNs), short oligonucleotides that contain unmethylated cytosine-guanine dinucleotides, MDP (muramyl dipeptide, a natural partial structure of bacterial peptidoglycan analogues, bacterial (mutant) toxins (Cholera toxin, CT; heat-labile entrotoxin, LT); endogenous human immunomodulators e.g., human granulocyte macrophage stimulating factor (HgM-CSF) and interleukins (IL-12, IL-2) (Sesardic and Dobbelaer (2004)). Aluminium hydroxide (Alhydrogel) or aluminium phosphate are preferred.

“Substantially free of adjuvant” in this context means that there there is less than 0.05% adjuvant, more preferably less than 0.025% adjuvant, even more preferably less than 0.001% adjuvant. In one embodiment, the combination therapy or vaccine may be completely free of adjuvant.

Preferred dose ranges for administration are 0.2 μg to 100 μg polysaccharide and 0.2μg to 100 pg OMV. Preferably 0.2 to 50 μg, more preferably 0.2 to 20 μg, more preferably 0.2 to 10 μg, more preferably 0.2 to 0.5 μg, most preferably about 0.5 μg. The ratio of OMVs to polysaccharide may be 1:1 to 20:1, preferably 1:1 to 10:1, preferably 2:1 to 8:1, more preferably 3:1 to 6:1, most preferably 5:1.

In further embodiments, the invention provides the use of the conjugates and OMVs as described herein in the manufacture of medicaments, e.g., for the treatment or prevention of the disorders discussed, such as infectious disease or cancer. Methods of treatment using the combination therapy and vaccines discussed above are also provided. In such methods of treatment an effective amount of the combination therapy or vaccine disclosed herein is administered to a patient.

Accordingly, in an embodiment, the present invention provides the use of a conjugate of a polysaccharide and a carrier protein (polysaccharide:protein conjugate) as defined herein in the manufacture of a combination therapy for the prevention or treatment of infectious disease or cancer, wherein said combination therapy further comprises outer membrane vesicles (OMVs) from commensal Neisseria as defined herein.

In a further embodiment, the invention provides the use of outer membrane vesicles (OMVs) from commensal Neisseria as defined herein in the manufacture of a combination therapy for the prevention or treatment of infectious disease or cancer, wherein said combination therapy further comprises a conjugate of a polysaccharide and a conjugate protein (polysaccharide:protein conjugate) as defined herein.

In the combination therapy, vaccine or use of the invention, it is preferred that the polysaccharide is from Neisseria meningitidis, preferably capsular polysaccharide from Neisseria meningitidis, most preferably from Neisseria meningitidis serogroup C. The preferred carrier protein is tetanus toxoid. It is preferred that the OMVs are from Neisseria lactamica.

In a most preferred embodiment, the capsular polysaccharide is from Neisseria meningitidis, the OMVs are from Neisseria lactamica, and the combination therapy, vaccine or use is for the treatment or prevention of meningococcal disease.

Aspects and embodiments of the invention will now be illustrated by the following examples, and with reference to the following Figures, in which:

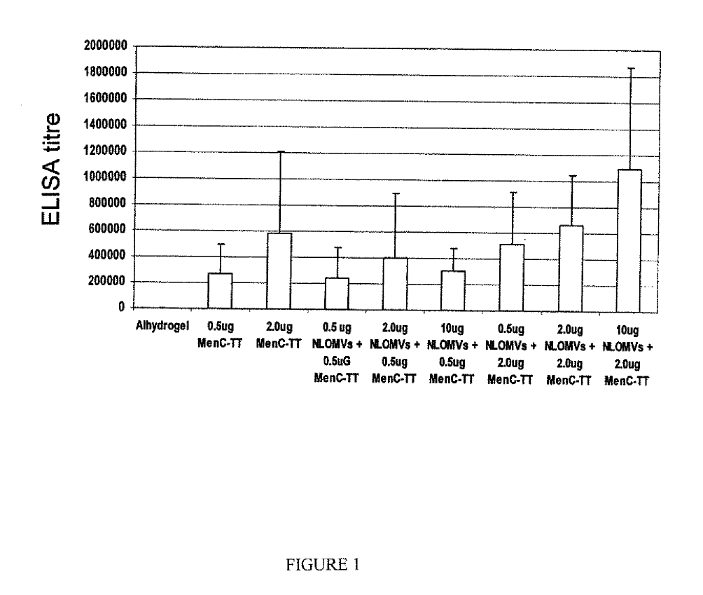

FIG. 1 shows the total IgG response in mice to N. meningitidis serogroup C polysaccharide as determined by ELISA. The graph shows the geometric mean titres against MenC polysaccharide (n=10). NLOMV=Neisseria lactamica OMVs. MenCTT=N. meningitidis serogroup C polysaccharide conjugated with Tetanus Toxin.

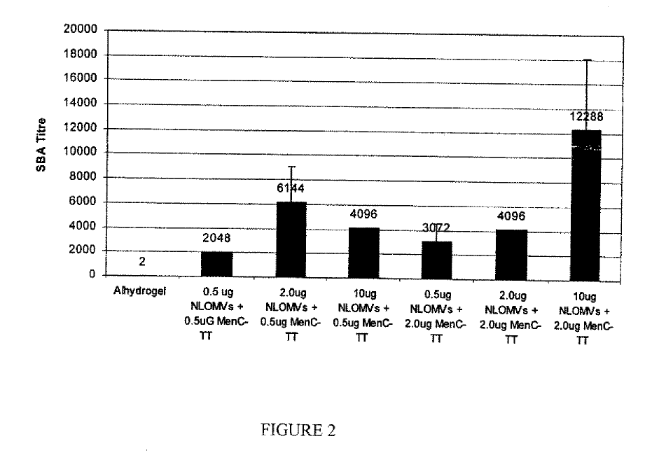

FIG. 2 shows the serum bactericidal response in mice against serogroup C target N. meningitidis. The graph shows the Mean +/−SD SBA (n=2) using pooled sera.

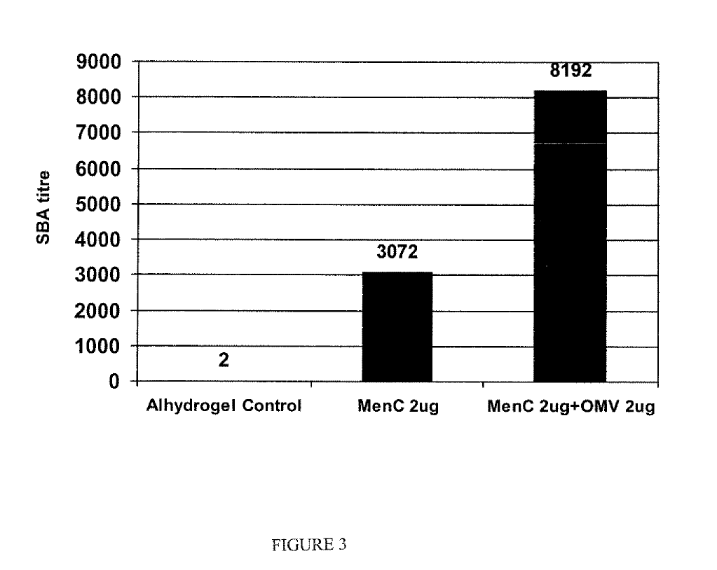

FIG. 3 shows a further experiment showing serum bactericidal response against serogroup C N. meningitidis.

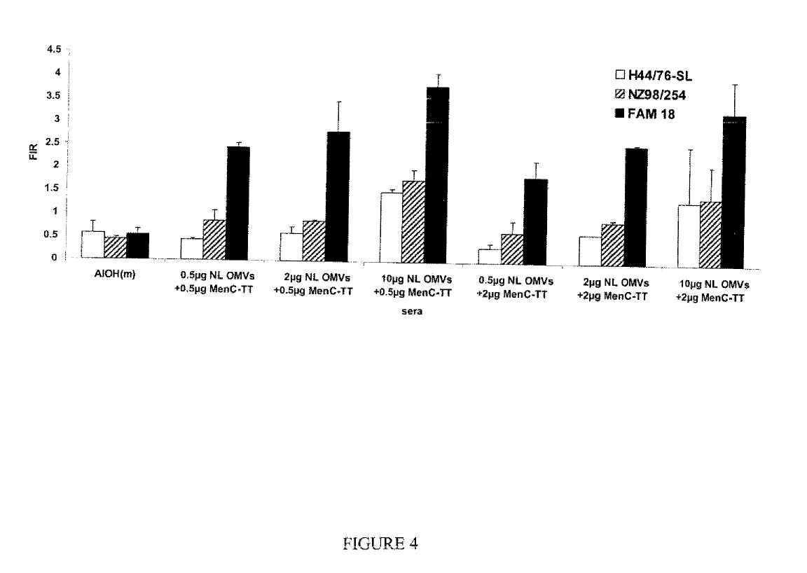

FIG. 4 shows the results of an osponophagocytosis assay using the MenC conjugates plus OMVs as shown the Figure. The assay is carried out against strains FAM18 (serogroup C), H44/76-SL (serogroup B) and NZ98/254 (serogroup B).

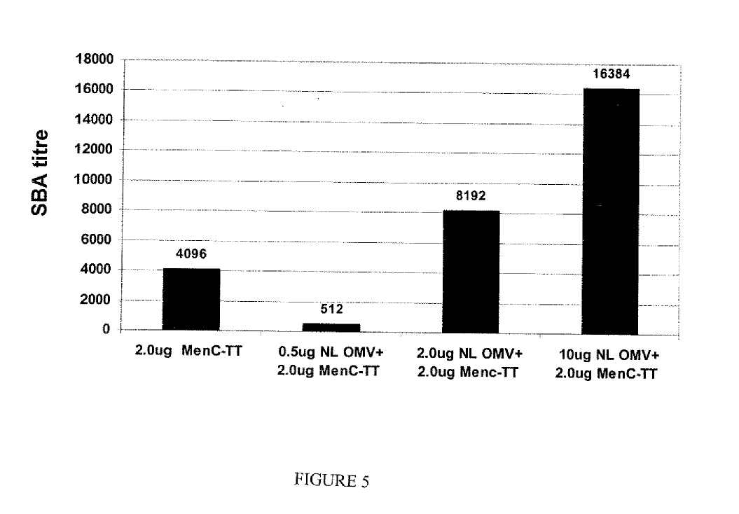

FIG. 5 shows further serum bactericidal assay titres for Men C conjugates plus OMVs.

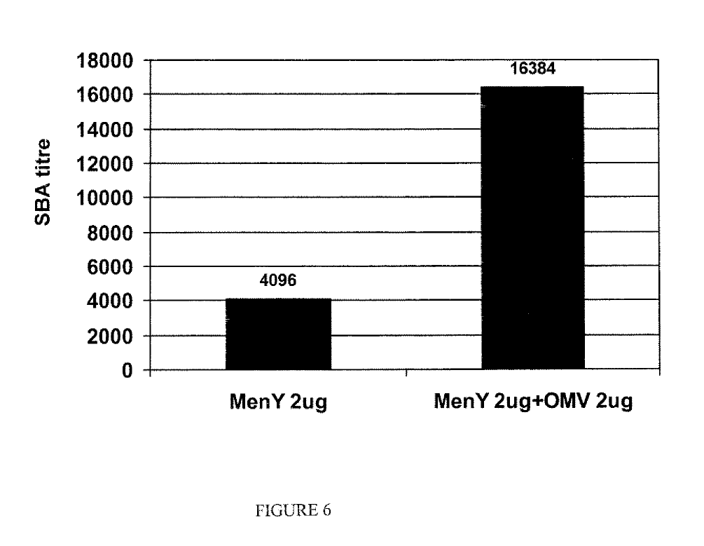

FIG. 6 shows serum bactericidal assay titres from Men Y conjugates plus OMVs.

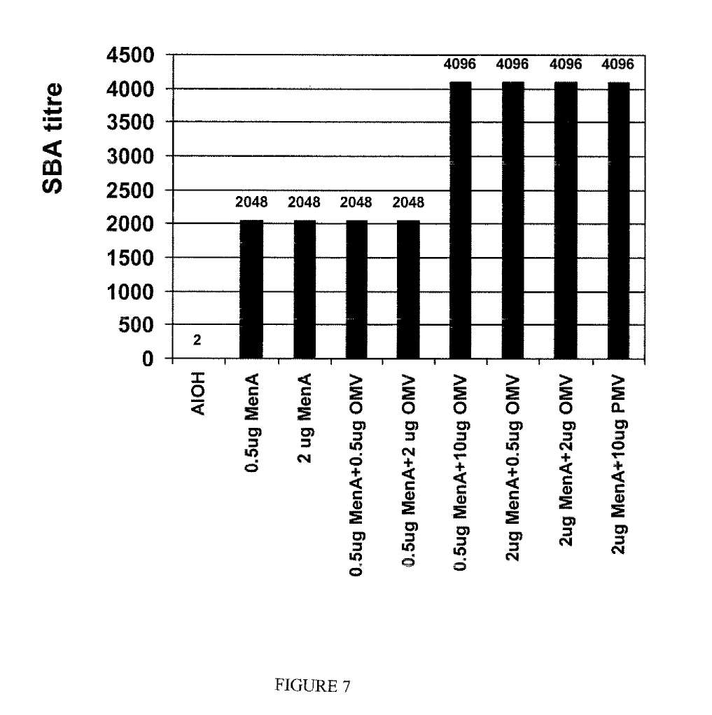

FIG. 7 shows serum bactericidal assay titres from Men Y conjugates plus OMVs.

### EXAMPLES

The following examples show that a mixture of OMVs isolated from Neisseria lactamica and capsular polysaccharide from N. meningitidis produced unexpectedly high titre responses to N. meningitidis in the serum bactericidal assay (SBA), which is the accepted serological correlate of protection (Snape and Pollard, 2005; Andrews et al 2003)).

N. lactamica Outer Membrane Vesicles (OMVs) and Adsorption to Adjuvant

Storage of Bacteria:

N. lactamica strain Y92-1009 was cultivated on tryptone soya agar +1.0% yeast extract and frozen as stock cultures in Frantz media (L-glutamic acid 1.6 g/L, L-cysteine 0.012 g/L, sodium di-hydrogen orthophosphate 2.5 g/L, potassium chloride 0.09 g/L, ammonium chloride 1.25 g/L, magnesium sulphate 0.6 g/L, glucose 5.0 g/L, yeast extract 2.0 g/L, 2 M sodium hydroxide as required pH 7.3, containing 30% (V/V) glycerol and stored at −70° C.

Isolation of Outer Membrane Vesicles:

For the production of OMVs, stock cultures were used to inoculate 12×10 ml Frantz media which were incubated in an orbital shaking incubator at 37° C. overnight. The overnight cultures were then used to inoculate 12×100 ml Frantz media which were then incubated as above for 6 hours. 75 ml of the 6 hour cultures were then transferred into 12×750 ml Frantz media which were incubated under the conditions described above for a further 18 hours. Outer membrane vesicles from N. lactamica were prepared by deoxycholate extraction as follows. The final cultures were centrifuged for 1 hour at 5000×g at a temperature between 4° C.-10° C., following this the cell paste was retained and the supernatant was discarded. The cell paste was re-suspended in buffer 1 (Tris-HCl 12.1 g/L, EDTA 3.72 g/L, deoxycholate acid sodium salt 5.0 g/L, 2 M sodium hydroxide as required pH 8.6, WFI water) to a ratio of 5:1 (V/W), homogenised and then centrifuged at 20,000×g for 30 minutes. The supernatant was discarded and the cell paste was re-suspended in buffer 1, with the above centrifuge step was repeated. The resulting supernatants were pooled and following homogenisation were ultracentrifuged at 125,000×g for 2 hours at 40° C. Following this the supernatant was discarded and the pellets were re-suspended in buffer 2 (Tris-HCl 6.05 g/L, EDTA 0.744 g/L, deoxycholate acid sodium salt 12 g/L, 2 M sodium hydroxide as required pH 8.6, WFI water), homogenised and ultracentrifuged as above. Finally the supernatant was discarded and the pellet re-suspended in buffer 3 (glycine 15.01 g/L, sucrose 30 g/L, 2 M sodium hydroxide as required pH 8, WFI water) at a concentration of 400 μg/ml. OMV bulk material was filter sterilized (double 0.2 μm pore size filter) and diluted to an appropriate protein concentration using buffer 3, prior to adsorption to aluminium hydroxide (Alhydrogel, Brenntag Biosector, Denmark) at a final concentration of 0.167% W/V. Protein concentrations were determined using an autoanalyser based on the Lowry method.

Production of Polysaccharide:Protein Conjugate

Neisseria meningitidis serotype C strain C11 polysaccharide (Yang and Jennings, 2001) was dissolved in phosphate buffer, and cooled below 15° C. NalO₄ was added to drive degradation of the polysaccharide to approximately 20 kD. This degraded and activated polysaccharide was concentrated and diafiltered using ultrafiltration. The activated polysaccharide was buffer-exchanged in 0.2M phosphate buffer pH 7.5 for conjugation.

Tetanus toxin (TT) (WHO, 1977; Document BLG/UNDP/77.2 Rev1) was detoxified with formaldehyde. TT was processed by diafiltration with 0.2M phosphate buffer pH 7.5 on 30 kD membrane.

The conjugation reaction was carried out by reacting the activated polysaccharide and concentrated TT in 3:1 ratio (75 mg/mL polysaccharide mixed with 25 mg/mL TT). Sodium cyanoborohydride was added to the mixture to concentration 30 mg/mL and incubated at 37° C. for two days. The reaction mixture was diafiltered on 100 kD membrane using 0.9% NaCl to give purified conjugate. The conjugate is referred to herein as MenC-TT.

Neisseria meningitidis serotype Y polysaccharide (PsY) was produced using a method based on (Yang and Jennings, 2001) PsY was then dissolved in 0.1M sodium phosphate buffer pH 7.5, and cooled below 15° C. NalO₄ was added to drive degradation and activation of the polysaccharide to approximately 20 kDa; at this stage 5% w/v Glycerol was added for quenching degradation reaction.

Tetanus toxin (TT) activation was achieved with Hydrazine-EDC. The activated TT (TTH) was buffer-exchanged in 3mM sodium carbonate saline for conjugation.

The conjugation reaction was carried out by reacting the activated polysaccharide and activated TT in 1:1 ratio (25 mg/mL polysaccharide mixed with 25 mg/mL TTH). This reaction mixture was then filtered through 0.22 u filter under sterile conditions. The reaction mixture was then incubated at room temperature for 36 h. On completion of conjugation based on HPLC profile, sodium borohydride was added to the mixture at 3 ul/mg of PsY to quench the conjugation. The reaction mixture was diafiltered on 100 kDa membrane using 0.9% NaCl to give purified conjugate.

Neisseria meningitidis serotype A polysaccharide (PsA) (Kshirsagar et al., 2007) was produced using a method based on WO05/014037. PsA was dissolved in HEPES buffer pH 7.5, and cooled below 15° C. NalO4 was added to drive degradation and activation of the polysaccharide to approximately 200 kDa; at this stage 5% w/v Glycerol was added to quench the degradation reaction. This degraded and activated polysaccharide was concentrated and diafiltered using HEPES-EDTA buffer pH 7.5. Tetanus toxin (TT) was activated with Hydrazine-EDC. The activated TT (TTH) was buffer-exchanged in 3 mM sodium carbonate saline for conjugation.

The conjugation reaction was carried out by reacting the activated polysaccharide and activated TT in 1:1 ratio (25 mg/mL polysaccharide mixed with 25 mg/mL TTH). This reaction mixture was filtered through 0.22 u filter under sterile conditions. The reaction mixture was then incubated at room temperature for 4 hr. On completion of conjugation based on HPLC profile, sodium borohydride was added to the mixture at 3 ul/mg of PsA for quenching the conjugation. The reaction mixture was diafiltered on 300 kD membrane using WFI. The conjugate is further purified by 30% ammonium sulphate precipitation. The precipitate is dissolved and extensively diafiltered on 300 KDa membrane using 20 mM Tris buffer pH 7.0.

Preparation of Combination of OMV+Conjugate Vaccines

A total volume of 7.5 ml of each vaccine was prepared by mixing the components (OMV, polysaccharide conjugate and Alhydrogel), as set out in the following tables:

For meningitidis Serogroup C (MenC):

| | Vol OMV | Vol MenC- | Vol 2% | Vol 0.2M |
|---|---|---|---|---|
| | (stock | TT (stock | Alhydrogel | glycine, 3% |
| | conc = | conc = | (Brenntag | sucrose pH |
| Vaccine | 410 μg/ml) | 687 μg/ml) | Biosector) | 8.0 buffer |
| 0.5 μg OMV + | 46 μl | 27 μl | 1.24 ml | 6.187 ml |
| 0.5 μg MenC- | | | | |
| TT | | | | |
| 2.0 μg OMV + | 183 μl | 27 μl | 1.24 ml | 6.05 ml |
| 0.5 μg MenC- | | | | |
| TT | | | | |
| 10 μg OMV + | 915 μl | 27 μl | 1.24 ml | 5.318 ml |
| 0.5 μg MenC- | | | | |
| TT | | | | |
| 0.5 μg OMV + | 46 μl | 109 μl | 1.24 ml | 6.105 ml |
| 2.0 μg MenC- | | | | |
| TT | | | | |
| 10 μg OMV + | 915 μl | 109 μl | 1.24 ml | 5.236 ml |
| 2.0 μg MenC- | | | | |
| TT | | | | |

For meningitidis Serogroup Y (MenY):

| | | | | | Vol 0.2M |
|---|---|---|---|---|---|
| | | Vol MenY- | Vol 2% | | glycine, 3% |
| | Vol OMV | TT (stock | Alhydrogel | | sucrose |
| | (stock conc = | conc = | (Brenntag | | pH 8.0 |
| Vaccine | 410 μg/ml) | 1 mg/ml) | Biosector) | Vol. PBS | buffer |
| 2.0 μg | 0 μl | 75 μl | 1.24 ml | 3.65 ml | 2.51 ml |
| MenY-TT | | | | | |
| 2.0 μg OMV + | 183 μl | 75 μl | 1.24 ml | 1.8 ml | 4.202 ml |
| 2.0 μg | | | | | |
| MenY-TT | | | | | |

For meningitidis Serogroup A (MenA):

12 ml volume totals were used.

| | | | | Vol 2% | | | Vol 0.2M | |
|---|---|---|---|---|---|---|---|---|
| | Vol OMV | | Vol MenA | Alhydrogel | | | glycine, 3% | |
| | (stock conc = | | (stock conc = | (Brenntag | | | sucrose pH | |
| Vaccine | 410 μg/ml) | | 209.4 μg/ml) | Biosector) | Vol. PBS | | 8.0 buffer | |
| 0.5 μg MenA | 0 | μl | 143.3 μl | 1.98 ml | 5.856 | ml | 4.02 | ml |
| 2.0 μg MenA | 0 | μl | 573.1 μl | 1.98 ml | 5.427 | | 4.02 | ml |
| 0.5 μg MenA + | 73.2 | μl | 143.3 μl | 1.98 ml | 2.927 | ml | 6.947 | ml |
| 0.5 μg OMV | | | | | | | | |
| 0.5 μg MenA + | 292.7 | μl | 143.3 μl | 1.98 ml | 2.927 | ml | 6.727 | ml |
| 2.0 μg OMV | | | | | | | | |
| 0.5 μg MenA + | 1463.4 | μl | 143.3 μl | 1.98 ml | 2.927 | ml | 5.486 | ml |
| 10 μg OMV | | | | | | | | |
| 2.0 μg MenA + | 73.2 | μl | 573.1 μl | 1.98 ml | 2.427 | ml | 6.947 | ml |
| 0.5 μg OMV | | | | | | | | |
| 2.0 μg MenA + | 1463.4 | μl | 573.1 μl | 1.98 ml | 2.427 | ml | 5.486 | ml |
| 10 μg OMV | | | | | | | | |

Immunological Tests

Animal Sera:

Mouse serum was raised using vaccine preparations as described above. NIH mice (6 to 8 weeks old) (Harlan) were immunized by subcutaneous injection on days 0, 21, and 28, 0.2 ml doses (0.1 ml at each of two sites) were administered (groups of between 5-10 mice) Terminal sera were collected on day 35.

ELISA:

ELISA was used to determine whether there was any interference between the individual components in the vaccine, as has been reported by Finn and Heath (2005) in the case of some conjugate vaccines. Men C polysaccharide antigen (Men C Ps) was mixed with methylated human serum albumin (mHSA) and adsorbed onto 96 well polystyrene microtitre plates by overnight incubation. After washing the plates to remove unbound Men C Ps, serial dilutions of test sera were applied to the plate along with serial dilutions of the reference and control sera. After appropriate incubation and washing, the bound antibodies on the plate specific for Men C Ps were detected using a goat anti-mouse IgG Fey chain-specific antibody conjugated to alkaline phosphatase. Finally, the addition of a suitable substrate causes a colour reaction proportional to the amount of bound antibody in each well. The colour reaction (absorbance) was measured at 405 nm and 690 nm as reference wavelength using an ELISA microplate reader. Test sera were assigned a titre value by using absorbance measurements of serial dilutions to interpolate values from the reference serum curve on each plate. The method is essentially as described by Gheesling et al. (1994). The results are shown in FIG. 1, show that high titre antibodies specific for Men C Ps were raised and that there is no interference between Men C Ps and the OMVs.

Serum Bactericidal Assay:

N. meningitidis Serogroup C:

Serum bactericidal activity (SBA) was determined as described by Maslanka et al. (1997) using N. meningitidis strain C11 and baby rabbit complement. Approximately 10 colonies of N. meningitidis strain C11 were subcultured onto a Columbia Blood Agar (CBA) plate and incubated for 4 h at 37° C. with 5% CO₂. After 4 h, bacteria were suspended in bactericidal buffer (Hanks balanced salt solution; Gibco, Paisley, United Kingdom) containing 0.5% bovine serum albumin (BSA) (Sigma, Poole, United Kingdom) and 0.5 U/ml heparin (CP Pharmaceuticals, Wrexham, United Kingdom) and adjusted to 8×104 organisms/ml. Equal volumes (10 μl) of the bacterial suspension and baby rabbit complement (Pelfreez, Rogers, Ark.) were added to 20 μl heat-inactivated test serum serially diluted twofold in bactericidal buffer in 96-well U-bottom microtiter plates (Greiner, Frickenhausen, Germany). The reaction mixture was mixed by gentle tapping, and the number of CFU at time zero was determined by allowing 10 pl of the reaction mixture (in the control column) to flow 8 to 10 cm, in lanes, down a CBA plate (the tilt method). Following incubation of the reaction mixture at 37° C. for 60 min, 10 μl was removed from each well and plated on CBA using the tilt method to determine the number of CFU per well after 60 min of incubation. Colonies were counted after overnight incubation at 37° C. with 5% CO₂. SBA titers were expressed as the reciprocal of the final serum dilution step giving ≧50% killing at 60 min compared to the number of CFU at time zero. The results are shown in FIGS. 2, 3 and 5 and show that the immune bactericidal response to N. meningitidis serogroup C is enhanced by the addition of Neisseria lactamica OMVs.

Similar experiments were carried out for serogroups A and Y and confirmed the findings with serogroup C. For N. meningitidis serogroup A, the serum bactericidal activity was carried out using N. meningitidis strain F8238 (A:4,21:P1:20,9) and for N. meningitidis serogroup Y using strain M01 242975 (Y:2a:P1.5.2). The assays were carried out using the method of Findlow et al (2006). The results are shown in FIGS. 6 and 7.

Opsonophagocytic Assay:

Bacterial strains (N. meningitidis serogroup C strain FAM18; N. meningitidis serogroup B strains H44/76 and NZ98/254) were grown to log phase in 10 mL Frantz medium, washed and resuspended in 1 ml phosphate buffered saline (PBS) containing 1 μg/mL BCECF/AM and incubated with vigorous shaking at 37° C. for 1 h. After washing, the bacteria were killed using 2% sodium azide for 24 h. 10 μl bacteria at 6.25×108/mL in OP buffer (Hanks Balanced Salt Solution containing Ca²⁺, Mg²⁺and 2% skimmed milk powder) was added to 10 μL baby rabbit complement and 20 μL of antibody that has been raised against the OMV/polysaccharide conjugate combinations as shown in FIG. 4 and as described above (at 1:10 in OP buffer) before incubation at 37° C. for 7.5 min with vigorous shaking. 50 μL of 5-day, DMF-differentiated HL60 cells at 2.5×107 in OP buffer were then added and incubation continued for a further 7.5 min. The reaction was stopped with the addition of 80 μL ice-cold PBS containing EDTA and the samples kept on ice until analysed. Immediately before flow-cytometric analysis 50 μl Trypan Blue solution (Sigma, UK) was added to quench external fluorescence. The fluorescence of 5000 HL60 cells was determined and a ratio of the mean fluorescence obtained with or without immune serum is calculated. The results are shown in FIG. 4. The black bar shows the opsonophagocytic activity generated by the MenC polysaccharide conjugate against the Men C strain FAM18. The white and hatched bars show that the N. lactamica OMVs generate some opsonophagocytic activity against the serogroup B N. meningitidis strains. This is because of cross-reactivity between N. lactamica OMVs and N. meningitidis, as has been previously described (WO00/50074).

### REFERENCES

Aaberge IS et al (2005) “Combined Administration of Meningococcal Serogroup B Outer Membrane Vesicle Vaccine and Conjugated Serogroup C Vaccine indicated for Prevention of Meningococcal Disease Is Safe and Immunogenic”. Clinical and Diagnostic Laboratory Immunology Vol 12, No. 5, May, p 599 - 605.

Andrews N et al (2003) “Validation of Serological Conjugate of Protection for Meningococcal C Conjuagate Vaccine by Using Efficacy Estimates from Postlicensure Surveillance in England. Clinical and Diagnostic Laboratory Immunology. Vol 10, No. 5. September, 2003 p 780-786.

Findlow et al (2006) Immunoglobulin G subclass response to a meningococcal quadrivalent polysaccharide-diphtheria toxoid conjugate vaccine. Clin Vaccine Immunol. 13(4):507-510.

Finn and Heath (2005) “Conjugate vaccines” Arch. Dis. Child. 90, 667-669.

Frasch C E, et al (2001). Outer membrane protein vesicle vaccines for meningococcal disease. In Methods in Molecular Medicine vol 66: Meningococcal Vaccines; Methods and Protocols. Pollard A J and Maiden M C J, eds. Humana Press, Totowa N J, p81-107.

Fukasawa L O et al (1999) “Neisseria meningitidis serogroup C polysaccharide and serogroup B outermembrane vesicle conjugate as a bivalent meningococcus vaccine candidate. Vaccine 17,2951-2958.

Gheesling, L. L., et al (1994). “Multicenter comparison of Neisseria meningitidis serogroup C anti-capsular polysaccharide antibody levels measured by a standardized enzyme-linked immunosorbent assay”. J. Clin. Microbiol. 32:1475-1482

Gorringe (2005) “Can Neisseria lactamica antigens provide an effective vaccine to prevent meningococcal disease?”. Expert Rev. Vaccines 4 (3), 373-379.

Harrison L. H (2006) Prospects for vaccine prevention of meningococcal infection. Clinical Microbiology Reviews19:142-64.

Jones et al (2005). “Vaccines based on the cell surface carbohydrates of pathogenic bacteria”. Annals of the Brazilian Academy of Sciences 77(2):, 293-324.

Kshirsagar et al (2007) “Safety, immunogenicity, and antibody persistence of a new meningococcal group A conjugate vaccine in healthy Indian adults”. Vaccine 25S, A-101-107.

Maslanka S E et al (1997) “Standardization and a multilaboratory comparison of Neisseria meningitidis serogroup A and C serum bactericidal assays”. The Multilaboratory Study Group. Clin. Diagn. Lab Immunol. March; 4(2), 156-67.

O'Dwyer C A, et al (2004) Expression of heterologous antigens in commensal Neisseria spp.: preservation of conformational epitopes with vaccine potential. Infection and Immunity.72: 6511-8.

Oliver K J et al (2002) Neisseria lactamica protects against experimental meningococcal infection. Infection and Immunity 70, 3621-3626.

Poolman J, Berthet FX. (2001) Alternative vaccine strategies to prevent serogroup B meningococcal diseases. Vaccine. 20 Suppl 1:S24-6.

Sardinas et al (2006) “Outer membrane vesicles of Neisseria lactamica as a potential mucosal adjuvant”. Vaccine 24, 206-214.

Sesardic and Dobbelaer (2004) “European Union regulatory developments for new vaccine adjuvants and delivery systems” Vaccine 22 2452-2456.

Sierra GVG et al (1991) “Vaccine against Group B Neisseria meningitidis: protection trial and mass vaccination results in Cuba”. NIPH Annals December 1991, Volume 14, Number 2 195-207.

Slovin et al (2005) “Carbohydrate vaccines as immunotherapy for cancer” Immunol. Cell. Biol. August 83(4), 418-28.

Snape M D and Pollard A J (2005) “Meningococcal polysaccharide-protein conjugate vaccines” Lancet Infect. Dis.5: p 21-30.

Stephens, D. S (2007) “Conquering the Meningococcus” FEMS Microbiol Rev 31, 3-14.

World Health Organisation. 1977. Document BLG/UNDP/77.2 Rev1. Manual for the production and control of vaccines: tetanus toxoids. World Health Organization.Geneva. Document BLG/UNDP/77.2 Rev1, from WHO Biologicals, Geneva

WO00/50074 International Patent Application, published on 31 August 2000.

WO 03/051379 International Patent Application, published on 26 June 2003

WO05/014037 International Patent Application, published on 17 February 2005

WO06/008504 International Patent Application, published on 26 January 2006

Yang Q and Jennings H. (2001). Purification of capsular polysaccharide. In: Methods in Molecular Medicine vol 66: Meningococcal Vaccines; Methods and Protocols. Pollard A J and Maiden M C J, eds. Humana Press, Totowa N.J., p41-47.

1. A combination therapy comprising:
(a) a conjugate of a polysaccharide and a carrier protein (polysaccharide:protein conjugate); and
(b) commensal Neisseria outer membrane vesicles (OMVs).

2. The combination therapy according to claim 1, wherein the polysaccharide is from Gram negative bacteria.

3. The combination therapy according to claim 1, wherein the polysaccharide is from Gram positive bacteria.

4. The combination therapy according to claim 1, wherein the polysaccharide is a capsular polysaccharide.

5. The combination therapy according to claim 1, wherein the polysaccharide is a lipopolysaccharide.

6. The combination therapy according to claim 2, wherein said polysaccharide is from a bacteria selected from the group consisting of: Escherichia coli, Francisella tularensis, Haemophilus influenzae, Klebsiella, Moraxella catarrhalis, Neisseria meningitidis, Porphyromonas gingivalis, Pseudomonas aeruginosa, Burkholderia cepacia, Salmonella typhi, Salmonella typhimurium, Salmonella paratyphi, Shigella dysenteriae, Shigella flexneri, Shegella sonnei and Vibrio cholera.

7. The combination therapy according to claim 3, wherein said polysaccharide is from a bacteria selected from the group consisting of: Enterococcus faecalis, Enterococcus faecium, Group A Streptococcus, Group B Streptococcus, Mycobacterium tuberculosis, Staphylococcus aureus, Staphylococcus epidermidis and Streptococcus pneumoniae.

8. The combination therapy according to claim 1, wherein the polysaccharide is from a eukaryotic cell.

9. The combination therapy according to claim 8, wherein the eukaryotic cell is a cell of a tumour, and wherein the tumour is selected from the group of consisting of: B cell lymphoma, breast cancer, colon cancer, lung cancer, melanoma, neuroblastoma, ovarian cancer, prostate cancer, sarcoma, small cell lung cancer, and stomach cancer.

10. The combination therapy according to claim 2, wherein the polysaccharide is from pathogenic Neisseria.

11. The combination therapy according to claim 10, wherein said polysaccharide is Neisseria meningitidis capsular polysaccharide.

12. The combination therapy according to claim 11, wherein said N. meningitidis is selected from N. meningitidis serogroups A, C, W135, B and Y.

13. The combination therapy according to claim 12, wherein said N. meningitidis is N. meningitidis serogroup C.

14. The combination therapy according to claim 2, wherein the polysaccharide is capsular polysaccharide from N. meningitidis serogroup A, C, W135 or Y.

15. The combination therapy according to claim 13, which further comprises a polysaccharide conjugate from Haemophilus influenzae or from Streptococcus pnerumoniae.

16-20. (canceled)

21. The combination therapy according to claim 1, wherein said commensal Neisseria is selected from the group consisting of Neisseria lactamica, Neisseria sicca, Neisseria cinerea, Neisseria subflava, Neisseria elongata, Neisseria flavescens, and Neisseria polysaccharea.

22. The combination therapy according to claim 21, wherein the commensal Neisseria is Neisseria lactamica.

23. The combination therapy according to claim 1, wherein the carrier protein is selected from the group consisting of a toxoid, keyhole limpet haemocyanin, fimbrae, albumin, CRM197 and Pseudomonas aeruginosa exotoxin A.

24. (canceled)

25. The combination therapy according to claim 23, wherein said toxoid is selected from tetanus toxoid or diphtheria toxoid.

26. (canceled)

27. The combination therapy according to claim 1, wherein said conjugate and said outer membrane vesicles are formulated for simultaneous administration.

28-29. (canceled)

30. The combination therapy according to claim 1, wherein said conjugate and said outer membrane vesicles are formulated for sequential administration.

31. A vaccine composition comprising a conjugate of a polysaccharide and a carrier protein (polysaccharide:protein conjugate); and outer membrane vesicles (OMVs) from commensal Neisseria.

32-35. (canceled)

36. The combination therapy of claim 1, wherein the combination therapy, further comprises an adjuvant.

37-44. (canceled)

45. The vaccine of claim 31, wherein the vaccine further comprises an adjuvant.

46. A method of preventing or treating an infectious disease in a subject, comprising administering to a subject in need of prevention or treatment a therapeutically effective amount of the combination therapy of claim 1.

47. The method according to claim 46, wherein the conjugate (a) and the commensal Neisseria OMVs (b) are administered simultaneously, sequentially or separately.

48. The method according to claim 47, wherein the conjugate (a) is a conjugate of a capsular polysaccharide from Neisseria meningitidis, wherein the commensal Neisseria OMVs are from Neisseria lactamica, and wherein the infectious disease is meningococcal disease.

49. The method according to claim 48, wherein the infectious disease is meningococcal meningitis or meningococcal septicaemia.

50. A method of preventing or treating cancer in a subject, comprising administering to a subject in need of prevention or treatment a therapeutically effective amount of the combination therapy of claim 1.

51. The method according to claim 50, wherein the conjugate (a) and the commensal Neisseria OMVs (b) are administered simultaneously, sequentially or separately.
